(19) United States
(12) Patent Application Publication
(10) Pub. No.: US 2009/0142400 A1
(43) Pub. Date: Jun. 4, 2009

(54) ANALGESIC COATED MEDICAL PRODUCT

(76) Inventors: Michael C. Hiles, Lafayette, IN (US); Paul J. Hall, Lafayette, IN (US)

Correspondence Address: WOODARD, EMHARDT, MORIARTY, MCNETT & HENRY LLP, 111 MONUMENT CIRCLE, SUITE 3700, INDIANAPOLIS, IN, 46204-5137, US

(21) Appl. No.: 12/130,432

(22) Filed: May 30, 2008

Related U.S. Application Data
(60) Provisional application No. 60/941,073, filed on May 31, 2007.

(51) Int. Cl. A61K 9/14; A61K 31/167; A61K 31/445; A61P 29/00

(52) U.S. Cl. 424/486; 424/484; 514/626; 514/330

(57) ABSTRACT

Described are medical products including an analgesic such as lidocaine, bupivicaine, or a mixture thereof. Such medical products can find use in pain relief, particularly after surgery, and can be applied to tissue or can be implanted within a patient. Also described are medical products including an extracellular matrix material, one or more analgesic agents, and a carrier effective to extend the release of the agent(s). Related methods of manufacture and use are also described.

### REFERENCE TO RELATED APPLICATION

The present application claims the benefit of U.S. Provisional Patent Application Ser. No. 60/941,073, filed May 31, 2007, entitled “ANALGESIC COATED MEDICAL PRODUCT” which is hereby incorporated by reference in its entirety.

### BACKGROUND

The present invention resides generally in the field of medical products and in particular aspects to medical products useful, for example, in pain relief.

As further background, a variety of extracellular matrix (ECM) materials have been proposed for use in medical grafting, cell culture, and other related applications. For instance, medical grafts and cell culture materials containing submucosa derived from small intestine, stomach or urinary bladder tissues, have been proposed. See, e.g., U.S. Pat. Nos. 4,902,508, 4,956,178, 5,281,422, 5,554,389, 6,099,567 and 6,206,931. In addition, Cook Biotech Incorporated, West Lafayette, Ind., currently manufactures a variety of medical products based upon small intestinal submucosa under the trademarks SURGISIS®, STRATASIS® and OASIS®.

Needs remain for improved and/or alternative medical products that can be used in a wide variety of medical applications. The present invention provides such medical products, as well as methods of preparing and using the same.

### SUMMARY

In one aspect, the present invention provides a medical product including a collagenous extracellular matrix (ECM) material and an analgesic selected from the group consisting of lidocaine, bupivacaine, or a mixture thereof. In preferred embodiments, the ECM material comprises submucosa of a warm-blooded vertebrate.

In another aspect, the present invention provides a medical product useful in pain relief. The medical product includes a sheet comprising small intestinal submucosa. The sheet incorporates an analgesic selected from the group consisting of lidocaine, bupivicaine, or a mixture thereof. Such a medical product finds use for pain relief after surgery, including surgery to correct physical defects of the body.

In another aspect, the present invention provides a method for preparing a medical product. The method includes providing a sheet of a collagenous extracellular matrix (ECM) material. An analgesic is applied to at least a portion of the ECM material. In certain embodiments, the analgesic is applied to at least a portion of a tissue contacting surface of the ECM material. In other embodiments, the analgesic is applied to the ECM material as a whole. In preferred embodiments, the ECM material comprises submucosa of a warm-blooded vertebrate.

In another aspect, the present invention provides a method for treating a patient. The method includes providing a sheet of a collagenous extracellular matrix (ECM) material. An analgesic is applied to at least a portion of the ECM material. In certain embodiments, the analgesic is applied to at least a portion of a tissue contacting surface of the ECM material. In other embodiments, the analgesic is applied to the ECM material as a whole. The medical product is applied to the patient so as to treat the patient. In preferred embodiments, the ECM material comprises submucosa of a warm-blooded vertebrate.

In another aspect, the present invention provides a medical product. The medical product includes a collagenous extracellular matrix (ECM) material, a carrier, and one or more analgesic agents. The carrier and the analgesic agent(s) are incorporated on or in the ECM material. The carrier is effective to extend the duration of release of the agent(s) from the ECM material. In certain embodiments, the medical product can further include a polymer film or layer over any portion of the ECM material containing the analgesic agent(s).

Additional embodiments as well as features and advantages of the invention will be apparent from the further descriptions herein.

### BRIEF DESCRIPTION OF THE DRAWINGS

FIG. 1 provides a perspective view of a medical product of the invention containing a sheet of a collagenous extracellular matrix (ECM) material including an analgesic coating.

FIG. 2 provides a perspective view of a medical producing of the invention containing multiple sheets of a collagenous extracellular matrix (ECM) material including an analgesic coating.

### DETAILED DESCRIPTION

As disclosed above, the present invention provides medical products useful in a wide variety of medical applications. Such medical products include a collagenous extracellular matrix (ECM) material and an analgesic, and preferably an analgesic selected from the group consisting of lidocaine, buvivacaine, or a mixture thereof.

For the purposes of promoting an understanding of the principles of the invention, reference will now be made to the embodiments illustrated in the drawings and specific language will be used to describe the same. It will nevertheless be understood that no limitation of the scope of the invention is thereby intended, and alterations and modifications in the illustrated medical products, and further applications of the principles of the invention as illustrated therein are herein contemplated as would normally occur to one skilled in the art to which the invention relates.

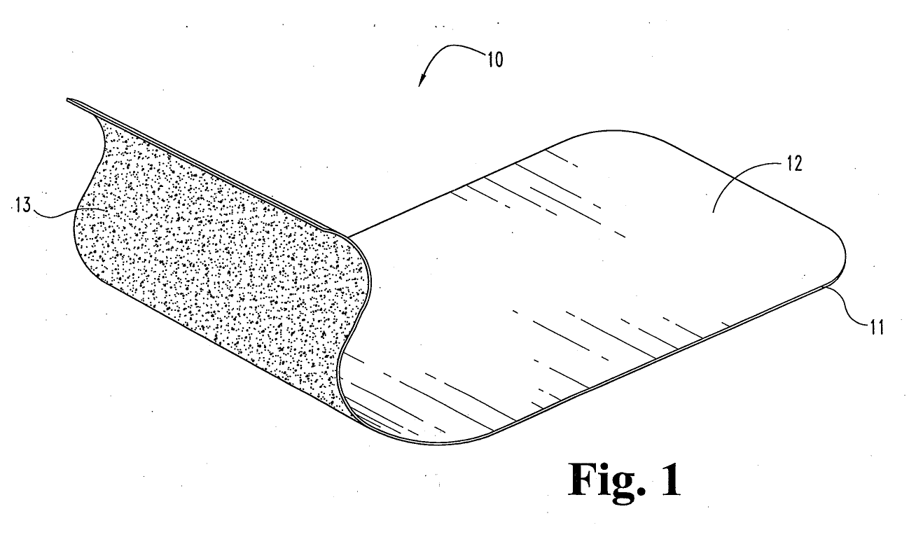

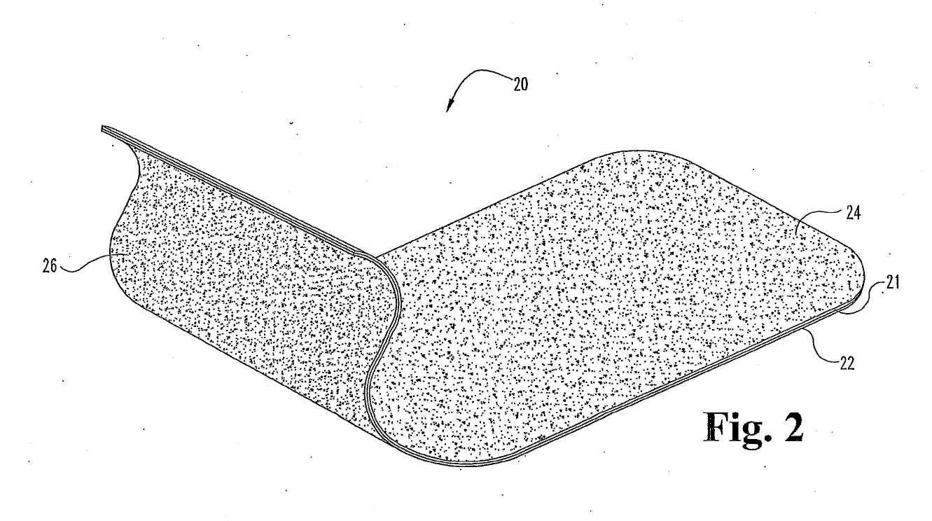

With reference now to FIG. 1, shown is a medical product 10 of the invention. The medical product 10 includes a sheet of a collagenous ECM material 11 having a first surface 12 and a second surface 13 opposing the first surface. The second surface 13 includes a coating comprising an analgesic selected from the group consisting of lidocaine, buvivacaine, or a mixture thereof. It will be understood that the first surface 12 can also include a coating comprising the analgesic(s), or that medical product 10 as a whole can incorporate the analgesic(s) generally homogenously in its structure. FIG. 2 illustrates a similar medical product 20 formed of multiple sheets 21 and 22 of a collagenous ECM material. The multiple sheets of a collagenous ECM material are bonded together to form a laminate. The first surface 24 of the laminate includes a coating comprising the analgesic(s) and the second surface 26 of the laminate includes a coating of the analgesic(s). In alternative embodiments, either the first surface 24 or the second surface 26 can include a coating of the analgesic(s). In still other embodiments, the laminate includes the analgesic(s) incorporated substantially homogenously in its structure, and/or in between sheets of the laminate.

Medical products of the invention, such as medical products 10 and 20, can be used to treat a variety of medical conditions, including those which cause pain to a patient. In this respect, a medical product of the invention can be used as a pain reliever and can either be applied to an external structure of a patient (e.g., skin) or can be implanted within a patient, for example to provide tissue support as in the case of hernia repair. Accordingly, a medical product of the invention can be configured in a variety of forms to suit its desired site of use. In preferred embodiments, a medical product of the invention is useful in surgery, for correction of physical defects of a patient. One advantage of using a collagenous ECM material in the medical products of the invention is that they can elute substances contained therein or thereon locally thereby eliminating or diminishing the need for systemic administration of the substance and minimizing the risk of systemic toxicity and adverse reactions.

Generally, when configured for application to tissue, the medical product of the invention is cut or otherwise configured to a desired size for its end use. In certain embodiments, the medical product is preferably sized larger than the tissue defect to which it is applied. Sizing the medical product in this way allows for easy attachment to the surrounding tissue.

In certain instances, it will be advantageous to securely attach the medical product to tissue or other structure. For example, once the sized medical product has been placed on, in, or around the area in need of treatment, the medical product can be more securely attached to the surrounding tissue or other structure using any of several known suitable attachment means. Suitable attachment means include, for example, stapling, suturing, bonding and the like. Preferably, the medical product is securely attached to the surrounding tissue or other structure by sutures or staples. There are a variety of synthetic materials currently available in the art for use as sutures. For example, sutures comprising Prolene™, Vicryl™, Mersilene™, Panacryl™, and Monocryl™, are contemplated for use in the invention. Other suture materials are well known to those skilled in the art. Medical adhesives as generally known in the art can also be used in conjunction with the medical products of the invention.

The medical product of the invention can be packaged in a dehydrated or hydrated state. Dehydration of a medical product of the invention can be achieved by any means known in the art. Preferably, dehydration is accomplished by either lyophilization or vacuum pressing and can simultaneously be utilized to bond layers of collagenous ECM material together. Any suitable solution can then optionally be used to rehydrate the medical product prior to use. Preferably, the rehydration solution comprises water or buffered saline. The above-described methods of dehydration and rehydration of the medical product allow for an effective shelf life and convenient packaging.

In certain embodiments, the medical product can be crosslinked. A medical product can be crosslinked once formed, or the collagenous ECM material can be crosslinked prior to applying the analgesic(s) to the material, or both. Increasing the amount (or number) of crosslinkages within the medical product and/or between two or more layers of the medical product can be used to enhance its strength. However, introduced crosslinkages within the medical product may also affect its remodelability. Consequently, in certain embodiments, the material used in a medical product will substantially retain its native level of crosslinking, or the amount of added crosslinkages within the material can be judiciously selected depending upon the desired treatment regime. In many cases, the material will exhibit remodelable properties such that the remodeling process occurs over the course of several days or several weeks. In preferred embodiments, the remodeling process occurs within a matter of about 5 days to about 12 weeks.

For use in the present invention, introduced crosslinking of the medical product may be achieved by photo-crosslinking techniques, or by the application of a crosslinking agent, such as by chemical crosslinkers, or by protein crosslinking induced by dehydration or other means. Chemical crosslinkers that may be used include for example aldehydes such as glutaraldehydes, diimides such as carbodiimides, e.g., 1-ethyl-3-(3-dimethylaminopropyl)carbodiimide hydrochloride, diisocyanates, such as hexamethylene-diisocyanate, ribose or other sugars, acyl-azide, sulfo-N-hydroxysuccinamide, or polyepoxide compounds, including for example polyglycidyl ethers such as ethyleneglycol diglycidyl ether, available under the trade name DENACOL EX810 from Nagese Chemical Co., Osaka, Japan, and glycerol polyglycerol ether available under the trade name DENACOL EX 313 also from Nagese Chemical Co. Typically, when used, polyglycerol ethers or other polyepoxide compounds will have from 2 to about 10 epoxide groups per molecule.

When multiple layers of a collagenous ECM material are used to form a laminate material, the layers of the laminate can be additionally crosslinked to bond multiple layers of a collagenous ECM material to one another. Thus, additional crosslinking may be added to individual layers prior to coupling to one another, during coupling to one another, and/or after coupling to one another.

It is advantageous to use a remodelable material for use in the medical products and methods of the present invention, and particular advantage can be provided by including a remodelable collagenous material. Such remodelable collagenous materials can be provided, for example, by collagenous materials isolated from a suitable tissue source from a warm-blooded vertebrate, and especially a mammal. Reconstituted or naturally-derived collagenous materials can be used in the present invention. Such materials that are at least bioresorbable will provide advantage in the present invention, with materials that are bioremodelable and promote cellular invasion and ingrowth providing particular advantage. Remodelable materials may be used in this context to promote cellular growth within the site in which a medical product of the invention is implanted. Moreover, the thickness of the medical product can be adjusted to control the extent of cellular ingrowth.

Suitable bioremodelable materials can be provided by collagenous extracellular matrix materials (ECMs) possessing biotropic properties, including in certain forms angiogenic collagenous extracellular matrix materials. For example, suitable collagenous materials include ECMs containing materials such as submucosa, renal capsule membrane, dermal collagen, dura mater, pericardium, fascia lata, serosa, peritoneum or basement membrane layers, including liver basement membrane. Suitable submucosa-containing materials for these purposes include, for instance, materials containing intestinal submucosa, including small intestinal submucosa, stomach submucosa, urinary bladder submucosa, and uterine submucosa.

The submucosa can be derived from any suitable organ or other biological structure, including for example submucosa derived from the alimentary, respiratory, intestinal, urinary or genital tracts of warm-blooded vertebrates. Submucosa useful in the present invention can be obtained by harvesting such tissue sources and delaminating the submucosa from smooth muscle layers, mucosal layers, and/or other layers occurring in the tissue source. For additional information as to submucosa useful in the present invention, and its isolation and treatment, reference can be made, for example, to U.S. Pat. Nos. 4,902,508, 5,554,389, 5,993,844, 6,206,931, and 6,099,567.

When a submucosa or other ECM material having differing characteristic sides is used in combination with a medical device, e.g., a surgical stapler, it can be oriented upon the medical device with a specified side directed outward for contact with the arm(s) of the surgical fastening device. For example, in the case of small intestinal submucosa, the material may be oriented with either the luminal or abluminal side facing outwardly for contact with the arm(s) of the surgical fastening device.

As prepared, the submucosa-containing material and any other ECM used may optionally retain growth factors or other bioactive components native to the source tissue. For example, the submucosa or other ECM may include one or more native growth factors such as basic fibroblast growth factor (FGF-2), transforming growth factor beta (TGF-beta), epidermal growth factor (EGF), and/or platelet derived growth factor (PDGF). As well, submucosa or other ECM used in the invention may include other biological materials such as heparin, heparin sulfate, hyaluronic acid, fibronectin and the like. Thus, generally speaking, the submucosal or other ECM material may include a native bioactive component that induces, directly or indirectly, a cellular response such as a change in cell morphology, proliferation, growth, protein or gene expression.

Submucosal or other ECM materials of the present invention can be derived from any suitable organ or other tissue source, usually sources containing connective tissues. The ECM materials processed for use in the invention will typically include abundant collagen, most commonly being constituted at least about 80% by weight collagen on a dry weight basis. Such naturally-derived ECM materials will for the most part include collagen fibers that are non-randomly oriented, for instance occurring as generally uniaxial or multi-axial but regularly oriented fibers. When processed to retain native bioactive components, the ECM material can retain these components interspersed as solids between, upon and/or within the collagen fibers. Particularly desirable naturally-derived ECM materials for use in the invention will include significant amounts of such interspersed, non-collagenous solids that are readily ascertainable under light microscopic examination. Such non-collagenous solids can constitute a significant percentage of the dry weight of the ECM material in certain inventive embodiments, for example at least about 1%, at least about 3%, and at least about 5% by weight in various embodiments of the invention.

The submucosal or other ECM material used in the present invention may also exhibit an angiogenic character and thus be effective to induce angiogenesis in a host engrafted with a device including the material. In this regard, angiogenesis is the process through which the body makes new blood vessels to generate increased blood supply to tissues. Thus, angiogenic materials, when contacted with host tissues, promote or encourage the formation of new blood vessels. Methods for measuring in vivo angiogenesis in response to biomaterial implantation have recently been developed. For example, one such method uses a subcutaneous implant model to determine the angiogenic character of a material. See, C. Heeschen et al., Nature Medicine 7 (2001), No. 7, 833-839. When combined with a fluorescence microangiography technique, this model can provide both quantitative and qualitative measures of angiogenesis into biomaterials. C. Johnson et al., Circulation Research 94 (2004), No. 2, 262-268.

Further, in addition or as an alternative to the inclusion of native bioactive components, non-native bioactive components such as those synthetically produced by recombinant technology or other methods, may be incorporated into the submucosa or other ECM tissue. These non-native bioactive components may be naturally-derived or recombinantly produced proteins that correspond to those natively occurring in the ECM tissue, but perhaps of a different species (e.g. human proteins applied to collagenous ECMs from other animals, such as pigs). The non-native bioactive components may also be drug substances. Illustrative drug substances that may be incorporated into and/or onto the ECM materials used in the invention include, for example, antibiotics, thrombus-promoting substances such as blood clotting factors, e.g. thrombin, fibrinogen, and the like. These substances may be applied to the ECM material as a premanufactured step, immediately prior to the procedure (e.g. by soaking the material in a solution containing a suitable antibiotic such as cefazolin), or during or after engraftment of the material in the patient. Alternatively or additionally, a non-native bioactive component can be included in the analgesic coating of the medical product. When included in the analgesic coating, the non-native bioactive component can be added at any point during preparation of the medical product, including being mixed with one or all of the analgesic coating components prior to application of the analgesic to a medical product or, alternatively, after the analgesic is formed and applied.

A non-native bioactive component can be applied to a submucosa or other ECM tissue by any suitable means. Suitable means include, for example, spraying, impregnating, dipping, etc. The non-native bioactive component can be applied to the ECM tissue either before or after the analgesic is applied to the material, or both. Similarly, if other chemical or biological components are included in the ECM tissue, the non-native bioactive component can be applied either before, in conjunction with, or after these other components.

Submucosa or other ECM tissue used in the invention is preferably highly purified, for example, as described in U.S. Pat. No. 6,206,931 to Cook et al. Thus, preferred ECM material will exhibit an endotoxin level of less than about 12 endotoxin units (EU) per gram, more preferably less than about 5 EU per gram, and most preferably less than about 1 EU per gram. As additional preferences, the submucosa or other ECM material may have a bioburden of less than about 1 colony forming units (CFU) per gram, more preferably less than about 0.5 CFU per gram. Fungus levels are desirably similarly low, for example less than about 1 CFU per gram, more preferably less than about 0.5 CFU per gram. Nucleic acid levels are preferably less than about 5 μg/mg, more preferably less than about 2 μg/mg, and virus levels are preferably less than about 50 plaque forming units (PFU) per gram, more preferably less than about 5 PFU per gram. These and additional properties of submucosa or other ECM tissue taught in U.S. Pat. No. 6,206,931 may be characteristic of the submucosa tissue used in the present invention.

In additional embodiments, medical products of the invention can include ECM's or other collagenous materials that have been subjected to processes that expand the materials. In certain forms, such expanded materials can be formed by the controlled contact of an ECM material with one or more alkaline substances until the material expands, and the isolation of the expanded material. Illustratively, the contacting can be sufficient to expand the ECM material to at least 120% of (i.e. 1.2 times) its original bulk volume, or in some forms to at least about two times its original volume. Thereafter, the expanded material can optionally be isolated from the alkaline medium, e.g. by neutralization and/or rinsing. The collected, expanded material can be used in any suitable manner in the preparation of a medical device. Illustratively, the expanded material can be enriched with bioactive components, dried, and/or molded, etc., in the formation of a graft construct of a desired shape or configuration. In certain embodiments, a medical graft material and/or device formed with the expanded ECM material can be highly compressible (or expandable) such that the material can be compressed for delivery, such as from within the lumen of a cannulated delivery device, and thereafter expand upon deployment from the device so as to become anchored within a patient and/or cause closure of a tract within the patient.

Expanded collagenous or ECM materials can be formed by the controlled contact of a collagenous or ECM material with an aqueous solution or other medium containing sodium hydroxide. Alkaline treatment of the material can cause changes in the physical structure of the material that in turn cause it to expand. Such changes may include denaturation of the collagen in the material. In certain embodiments, it is preferred to expand the material to at least about three, at least about four, at least about 5, or at least about 6 or even more times its original bulk volume. The magnitude of the expansion is related to several factors, including for instance the concentration or pH of the alkaline medium, exposure time, and temperature used in the treatment of the material to be expanded.

ECM materials that can be processed to make expanded materials can include any of those disclosed herein or other suitable ECM's. Typical such ECM materials will include a network of collagen fibrils having naturally-occurring intramolecular cross links and naturally-occurring intermolecular cross links. Upon expansion processing as described herein, the naturally-occurring intramolecular cross links and naturally-occurring intermolecular cross links can be retained in the processed collagenous matrix material sufficiently to maintain the collagenous matrix material as an intact collagenous sheet material; however, collagen fibrils in the collagenous sheet material can be denatured, and the collagenous sheet material can have an alkaline-processed thickness that is greater than the thickness of the starting material, for example at least 120% of the original thickness, or at least twice the original thickness.

Illustratively, the concentration of the alkaline substance for treatment of the remodelable material can be in the range of about 0.5 to about 4 M, with a concentration of about 1 M to about 3 M being more preferable. Additionally, the pH of the alkaline substance can in certain embodiments range from about 8 to about 14. In preferred aspects, the alkaline substance will have a pH of from about 10 to about 14, and most preferably of from about 12 to about 14.

In addition to concentration and pH, other factors such as temperature and exposure time will contribute to the extent of expansion, as discussed above. In this respect, in certain variants, the exposure of the collagenous material to the alkaline substance is performed at a temperature of about 4 to about 45° C. In preferred embodiments, the exposure is performed at a temperature of about 25 to about 40° C., with 37° C. being most preferred. Moreover, the exposure time can range from at least about one minute up to about 5 hours or more. In some embodiments, the exposure time is about 1 to about 2 hours. In a particularly preferred embodiment, the collagenous material is exposed to a 3 M solution of NaOH having a pH of 14 at a temperature of about 37° C. for about 1.5 to 2 hours. Such treatment results in collagen denaturation and a substantial expansion of the remodelable material. Denaturation of the collagen matrix of the material can be observed as a change in the collagen packing characteristics of the material, for example a substantial disruption of a tightly bound collagenous network of the starting material. A non-expanded ECM or other collagenous material can have a tightly bound collagenous network presenting a substantially uniform, continuous surface when viewed by the naked eye or under moderate magnification, e.g. 100× magnification. Conversely, an expanded collagenous material can have a surface that is quite different, in that the surface is not continuous but rather presents collagen strands or bundles in many regions that are separated by substantial gaps in material between the strands or bundles when viewed under the same magnification, e.g. about 100×. Consequently, an expanded collagenous material typically appears more porous than a corresponding non-expanded collagenous material. Moreover, in many instances, the expanded collagenous material can be demonstrated as having increased porosity, e.g. by measuring for an increased permeability to water or other fluid passage as compared to the non-treated starting material. The more foamy and porous structure of an expanded ECM or other collagenous material can allow the material to be cast or otherwise prepared into a variety of sponge or foam shapes for use in the preparation of medical materials and devices. It can further allow for the preparation of constructs that are highly compressible and which expand after compression. Such properties can be useful, for example, when the prepared medical graft material is to be compressed and loaded into a deployment device (e.g. a lumen thereof) for delivery into a patient, and thereafter deployed to expand at the implant site.

After such alkaline treatments, the material can be isolated from the alkaline medium and processed for further use. Illustratively, the collected material can be neutralized and/or rinsed with water to remove the alkalinity from the material, prior to further processing of the material to form a medical product of the invention.

A starting ECM material (i.e., prior to treatment with the alkaline substance) can optionally include a variety of bioactive or other non-collagenous components including, for example, growth factors, glycoproteins, glycosaminoglycans, proteoglycans, nucleic acids, and lipids. Treating the material with an alkaline substance may reduce the quantity of one, some or all of such non-collagenous components contained within the material. In certain embodiments, controlled treatment of the remodelable material with an alkaline substance will be sufficient to create a remodelable collagenous material which is substantially devoid of nucleic acids and lipids, and potentially also of growth factors, glycoproteins, glycosaminoglycans, and proteoglycans. This may be true for other processing techniques as discussed herein, such as the controlled treatment of the material with a detergent.

In certain embodiments, one or more bioactive components, exogenous or endogenous, for example, similar to those removed from an ECM material (i.e., both non-expanded and expanded materials) during processing, can be returned to the material. For example, an ECM material can include a collagenous material which has been depleted of nucleic acids and lipids, but which has been replenished with growth factors, glycoproteins, glycosaminoglycans, and/or proteoglycans. These bioactive components can be returned to the material by any suitable method. For instance, in certain forms a tissue extract, such as is discussed in U.S. Pat. No. 6,375,989, containing these components can be prepared and applied to an ECM collagenous material. In one embodiment, the ECM can be incubated in a tissue extract for a sufficient time to allow bioactive components contained therein to associate with the ECM material. The tissue extract may, for example, be obtained from non-expanded collagenous tissue of the same type used to prepare the expanded material. Other means for returning or introducing bioactive components to an ECM material include spraying, impregnating, dipping, etc. as known in the art. By way of example, an ECM material may be modified by the addition of one or more growth factors such as basic fibroblast growth factor (FGF-2), transforming growth factor beta (TGF beta), epidermal growth factor (EGF), platelet derived growth factor (PDGF), and/or cartilage derived growth factor (CDGF). As well, other biological components may be added to an ECM material, such as heparin, heparin sulfate, hyaluronic acid, fibronectin and the like. Thus, generally speaking, an ECM material may include a bioactive component that induces, directly or indirectly, a cellular response such as a change in cell morphology, proliferation, growth, protein or gene expression similar to a non-expanded collagenous material.

With respect to the above, a medical material can be provided in any suitable form prior to application of an analgesic thereto. Suitable forms include, for example, as one or more sheets or layers, as a foam, or as a sponge. The form used will typically depend on a variety of factors including, but not limited to, the end use of the medical product and the type of material used (e.g., synthetic or biological).

In embodiments of the invention where a medical product is provided in sheet form, the medical product will have a thickness in the range of about 50 to about 1000 microns, more preferably about 100 to 600 microns, and most preferably about 100 to about 350 microns. The medical product will desirably exhibit a suture retention strength in the range of about 100 to about 1000 gram force, e.g. typically in the range of about 200 to about 600 gram force, each of these based upon 5-0 Prolene suture and a bite depth of 2 mm. If needed to achieve these strengths, multiple layers of ECM collagenous tissue can be bonded together in any suitable fashion, including dehydrothermal bonding under heated, non-heated or lyophilization conditions, using adhesives as described herein, glues or other bonding agents, crosslinking with chemical agents or radiation (including UV radiation), or any combination of these with each other or other suitable methods.

Turning now to a discussion of the analgesic(s), said analgesic(s) can be applied to at least a portion of a collagenous ECM material by any suitable means. Suitable means include, for example, brushing, spraying, soaking, dipping, etc. In certain forms, a substantial portion of a surface of a medical material is coated with the adhesive. By “substantial portion” is meant that at least about 75% of a surface of a medical material is coated with an analgesic(s). The analgesic(s) can be applied alone or in combination with a suitable carrier. In certain embodiments, the analgesic(s) is applied in conjunction with a biodegradable macro-molecular substance such as a biodegradable polymer. Preferred such polymers include polylactic acid, polyglycolic acid, or copolymers thereof (PLGA). In one mode, to apply the polymer and analgesic(s), a solution of the substances can be prepared in a solvent and the ECM device soaked in the solution, e.g. to allow a precipitate containing the polymer and analgesic(s) to be deposited upon and/or within the device. The device can then be removed from the solution and further processed, e.g. dried, packaged and sterilized. In other embodiments, the analgesic(s) can be applied in conjunction with a biocompatible tissue adhesive. Preferred tissue adhesives include, for example, biodegradable polymer hydrogels such as FOCALSEAL® (biodegradable eosin-PEG-lactide hydrogel requiring photopolymerization with Xenon light wand) produced by Focal; or natural polymer adhesives such as those containing fibrogen, e.g. BERIPLAST® produced by Adventis-Bering; VIVOSTAT® produced by ConvaTec (Bristol-Meyers-Squibb); SEALAGEN™ produced by Baxter; FIBRX® (containing virally inactivated human fibrinogen and inhibited-human thrombin) produced by CyoLife; TISSEEL® (fibrin glue composed of plasma derivatives from the last stages in the natural coagulation pathway where soluble fibrinogen is converted into a solid fibrin) and TISSUCOL® produced by Baxter; QUIXIL® (Biological Active Component and Thrombin) produced by Omrix Biopharm; those comprising polyethylene glycol and/or collagen, such as a PEG-collagen conjugate produced by Cohesion (Collagen); or those containing cyanoacrylate such as HYSTOACRYL® BLUE (ENBUCRILATE) (cyanoacrylate) produced by Davis & Geek; NEXACRYL™ (N-butyl cyanoacrylate), NEXABOND™, NEXABOND™ S/C, and TRAUMASEAL™ (product based on cyanoacrylate) produced by Closure Medical (TriPoint Medical); DERMABOND™ which consists of 2-Octyl Cyanoacrylate produced by Dermabond (Ethicon); TISSUEGLU® produced by Medi-West Pharma; and VETBOND™ which consists of n-butyl cyanoacrylate produced by 3M. The biodegradable or biocompatible adhesive can be self-crosslinking (not requiring energy application for curing), or may require energy, such as radiation, for curing. The biodegradable macro-molecular substance and/or biocompatible tissue adhesive can be effective to extend the time of release of the analgesic(s) from the ECM material.

In certain embodiments, it may be desirable to better protect an analgesic agent(s) that has been coated onto at least a portion of a surface of a collagenous ECM material from aqueous or turbulent environments, such as may be encountered when a medical product is implanted into a patient. To accomplish this, an analgesic is provided to an ECM material as discussed herein. A synthetic or natural polymer solution can then be applied to any portion of the ECM material containing the analgesic(s), or applied over the ECM material as a whole, to mechanically entrap the analgesic(s) onto or within the ECM material. For example, an ECM material including an analgesic(s) can be dipped into a polymer solution. The polymer solution can then be allowed to polymerize to form a polymer film or layer over the analgesic(s), thus entrapping the analgesic(s) onto or within the ECM material. In other embodiments, a polymer solution can be painted or sprayed over the ECM material including an analgesic to provide a film or layer over the surface of the ECM material for entrapping the analgesic(s) onto or within the ECM material. Similarly, a pre-cast polymer film or layer can be coupled to the ECM material including an analgesic by sewing, adhering, or otherwise attaching the film or layer to a surface of the ECM material including an analgesic. Synthetic polymers useful for entrapping an analgesic to an ECM material can include, for example, polyhydroxyalkanoates, polycaprolactones, etc. Natural polymers useful for entrapping an analgesic to an ECM material can include, for example, fibrin, collagen, etc.

An analgesic as used herein preferably includes lidocaine, bupivacaine, or a mixture thereof, all of which are commercially available. The analgesic can be included in the medical product in an amount of about 0.01-0.5% (w/v), and preferably in an amount of about 0.1-0.2% (w/v). It will be understood that the amount of analgesic(s) can be varied to achieve the desired level of treatment.

The medical products of the invention can be provided in sterile packaging suitable for medical products. Sterilization may be achieved, for example, by irradiation, ethylene oxide gas, or any other suitable sterilization technique, and the materials and other properties of the medical packaging will be selected accordingly.

The use of the terms “a” and “an” and “the” and similar referents in the context of describing the invention (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Of course, variations of those preferred embodiments will become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context. In addition, all publications cited herein are indicative of the abilities of those of ordinary skill in the art and are hereby incorporated by reference in their entirety as if individually incorporated by reference and fully set forth.

What is claimed is:

1. A medical product, comprising:
a collagenous extracellular matrix (ECM) material including an analgesic selected from the group consisting of lidocaine, bupivacaine, or a mixture thereof.

2. The medical product of claim 1, wherein said collagenous extracellular matrix is in sheet form having a tissue contacting surface and a surface opposing said tissue contacting surface.

3. The medical product of claim 2, wherein at least a portion of said tissue contacting surface is coated with said analgesic.

4. The medical product of any of claims 1, wherein said ECM comprises submucosa.

5. The medical product of claim 4, wherein said submucosa is intestinal, urinary bladder or stomach submucosa.

6. The medical product of claim 5, wherein said submucosa is small intestinal submucosa (SIS).

7. A medical product useful in pain relief, comprising:
a sheet of small intestinal submucosa having a tissue contacting surface and a surface opposing said tissue contacting surface; and
an analgesic coating on at least a portion said tissue contacting surface, wherein said analgesic is selected from the group consisting of lidocaine, bupivacaine, or a mixture thereof.

8. The medical product of claim 7, wherein said medical product further includes a polymer layer to entrap said analgesic onto or within said sheet of small intestinal submucosa.

9. The medical product of claim 7, wherein said analgesic further comprises a carrier.

10. A method of preparing a medical product, comprising:
providing a sheet of a collagenous extracellular matrix (ECM) material, said sheet having a tissue contacting surface and a surface opposing said tissue contacting surface; and
applying an analgesic to at least a portion of the tissue contacting surface.

11. The method of claim 10, wherein said analgesic is applied to a substantial portion of the tissue contacting surface.

12. The method of claim 11, wherein said analgesic is applied generally homogenously throughout the medical product as a whole.

13. The method of claim 10, wherein said method further comprises applying a polymer onto at least the portion of the collagenous ECM material containing the analgesic, wherein said polymer is allowed to polymerize to form a polymer layer over said analgesic.

14. The method of claim 10, wherein said ECM comprises submucosa.

15. The method of claim 14, wherein said submucosa is intestinal, urinary bladder or stomach submucosa.

16. The method of claim 15, wherein said submucosa is small intestinal submucosa (SIS).

17. A method of treating a patient, comprising:
providing a sheet of a collagenous extracellular matrix (ECM) material, said sheet having a tissue contacting surface and a surface opposing said tissue contacting surface;
applying an analgesic to at least a portion of the tissue contacting surface to form a medical product; and
applying said medical product to the patient.

18. The method of claim 17, wherein said analgesic is applied to a substantial portion of the tissue contacting surface.

19. The method of claim 18, wherein said analgesic is applied generally homogenously throughout the medical product as a whole.

20. The method of claim 17, wherein said method further comprises applying a polymer onto at least the portion of the collagenous ECM material containing the analgesic, wherein said polymer is allowed to polymerize to form a polymer layer over said analgesic.

21. The method of claim 17, wherein said ECM comprises submucosa.

22. The method of claim 21, wherein said submucosa is intestinal, urinary bladder or stomach submucosa.

23. The method of claim 22, wherein said submucosa is small intestinal submucosa (SIS).

24. A medical product, comprising:
a collagenous extracellular matrix (ECM) material;
a carrier;
one or more analgesic agents;
wherein said carrier and analgesic agent(s) are incorporated on or in said ECM material; and
wherein said carrier is effective to extend the duration of release of said agent(s) from the ECM material.

25. The medical product of claim 24, wherein the ECM material is remodelable.

26. The medical product of claim 25, wherein the ECM material exhibits an angiogenic

27. The medical product of claim 24, wherein said product further comprises a polymer film over at least a portion of the ECM material containing the analgesic agent(s).
